(19) United States
(12) Patent Application Publication
(10) Pub. No.: US 2014/0172034 A1
(43) Pub. Date: Jun. 19, 2014

(54) INTRA-CARDIAC IMPLANTABLE MEDICAL DEVICE WITH IC DEVICE EXTENSION FOR LV PACING/SENSING

(71) Applicant: PACESETTER, INC., Sylmar, CA (US)

(72) Inventors: Gene A. Bornzin, Simi Valley, CA (US); John W. Poore, South Pasadena, CA (US); Zoltan Somogyi, Simi Valley, CA (US); Xiaoyi Min, Camarillo, CA (US); Didier Theret, Porter Ranch, CA (US)

(73) Assignee: PACESETTER, INC., Sylmar, CA (US)

(21) Appl. No.: 13/718,536

(22) Filed: Dec. 18, 2012

(51) Int. Cl. A61N 1/362; A61N 1/365

(52) U.S. Cl. CPC A61N 1/362; A61N 1/365
USPC 607/17; 607/9

(57) ABSTRACT

An assembly is provided for introducing a device within a heart of a patient. The assembly is comprised of a sheath having at least one internal passage. An intra-cardiac implantable medical device (IIMD) is retained within the at least one internal passage, wherein the IIMD is configured to be discharged from a distal end of the sheath. The IIMD has a housing with a first active fixation member configured to anchor the IIMD at a first implant location within a local chamber of the heart.

### FIELD OF THE INVENTION

Embodiments of the present invention generally relate to intra-cardiac implantable devices and methods for implanting the same. Embodiments more particularly relate to intra-cardiac implantable medical devices that utilize an IC device extension to afford dual chamber functionality.

### BACKGROUND OF THE INVENTION

Currently, permanently-implanted pacemakers (PPMs) utilize one or more electrically-conductive leads (which traverse blood vessels and heart chambers) in order to connect a canister with electronics and a power source (the can) to electrodes affixed to the heart for the purpose of electrically exciting cardiac tissue (pacing) and measuring myocardial electrical activity (sensing). These leads may experience certain limitations, such as incidences of venous stenosis or thrombosis, device-related endocarditis, lead perforation of the tricuspid valve and concomitant tricuspid stenosis; and lacerations of the right atrium, superior vena cava, and innominate vein or pulmonary embolization of electrode fragments during lead extraction. Further, conventional pacemakers with left ventricle (LV) pacing/sensing capability require multiple leads and a complex header on the pacemaker.

A small sized PPM device has been proposed with leads permanently projecting through the tricuspid valve and that mitigate the aforementioned complications. This PPM is a reduced-size device, termed a leadless pacemaker (LLPM) that is characterized by the following features: electrodes are affixed directly to the “can” of the device; the entire device is attached to the heart; and the LLPM is capable of pacing and sensing in the chamber of the heart where it is implanted.

LLPM devices, that have been proposed thus far, offer limited functional capability. These LLPM devices are able to sense in one chamber and deliver pacing pulses in that same chamber, and thus offer single chamber functionality. For example, an LLPM device that is located in the right atrium would be limited to offering AAI mode functionality. An AAI mode LLPM can only sense in the right atrium, pace in the right atrium and inhibit pacing function when an intrinsic event is detected in the right atrium within a preset time limit. Similarly, an LLPM device that is located in the right ventricle would be limited to offering VVI mode functionality. A VVI mode LLPM can only sense in the right ventricle, pace in the right ventricle and inhibit pacing function when an intrinsic event is detected in the right ventricle within a preset time limit.

It has been proposed to implant sets of multiple LLPM devices within a single patient, such as one or more LLPM devices located in the right atrium and one or more LLPM devices located in the right ventricle. The atrial LLPM devices and the ventricular LLPM devices wirelessly communication with one another to convey pacing and sensing information there between to coordinate pacing and sensing operations between the various LLPM devices.

However, these sets of multiple LLPM devices experience various limitations. For example, each of the LLPM devices must expend significant power to maintain the wireless communications links. The wireless communications links should be maintained continuously in order to constantly convey pacing and sensing information between, for example, atrial LLPM device(s) and ventricular LLPM device(s). This pacing and sensing information is necessary to maintain continuous synchronous operation, which in turn draws a large amount of battery power.

Further, it is difficult to maintain a reliable wireless communications link between LLPM devices. The LLPM devices utilize low power transceivers that are located in a constantly changing environment within the associated heart chamber. The transmission characteristics of the environment surrounding the LLPM device change due in part to the continuous cyclical motion of the heart and change in blood volume. Hence, the potential exists that the communications link is broken or intermittent.

### SUMMARY

In accordance with one embodiment, an assembly is provided for introducing a device within a heart of a patient. The assembly is comprised of a sheath having at least one internal passage, wherein the sheath is configured to be maneuvered into a local chamber of the heart. An intra-cardiac implantable medical device (IIMD) is retained within the at least one internal passage, wherein the IIMD is configured to be discharged from a distal end of the sheath. The IIMD has a housing with a first active fixation member configured to anchor the IIMD at a first implant location within a local chamber of the heart. A first electrode is provided on the housing at a first position such that, when the IIMD is implanted in the local chamber, the first electrode is configured to engage wall tissue at a first activation site within a conduction network of a first chamber. An intra-cardiac (IC) device extension has a transition segment and an extension body. The transition segment electrically is coupled to the IIMD housing and the extension body. The transition segment is sufficient in length to enable the extension body to be spaced apart from the housing of the IIMD and is located in at least one of a coronary sinus and a tributary vein branching from the coronary sinus. The extension body is sufficient in length to extend along the at least one of the coronary sinus and tributary vein proximate to a second chamber of the heart. The extension body includes an active segment configured to be positioned at a second implant location proximate to the second chamber when the extension body is located at a desired position. A second electrode is provided on the active segment of the extension body. The second electrode is configured to engage wall tissue at a second activation site within the conduction network of the second chamber controller, within the housing, is configured to cause stimulus pulses to be delivered through at least one of the first and second electrodes to at least one of the first and second activation sites, respectively.

The sheath comprises a flexible, longitudinal, cylindrical open-ended tube defining the internal passage. The assembly may further comprise a pusher rod within the sheath, the pusher rod being removably connected to the IIMD, wherein the pusher rod is configured to push the IIMD out of the sheath and rotate the IIMD to actively attach the IIMD at the first implant location.

The sheath may include first and second lumens configured to receive the IIMD and the IC device extension, respectively. The extension body of the IC device extension may include a lumen therein with an open proximal end. The assembly may further comprise a placement tool received in the lumen to guide the extension body to the second implant location. Optionally, the extension body and placement tool may represent one of: i) a guide wire that passes through the lumen in the extension body and projects beyond an open distal end of the extension body; and ii) a stylet the projects into the lumen in the extension body and abuts against a closed distal end of the extension body. Optionally, the extension body may include a distal end having a flange thereon with a guide wire passage through the flange, the flange dimensioned to abut against and block a stylet when inserted into the lumen, the passage dimension to pass a guide wire therethrough when inserted into the lumen.

The IIMD may be anchored in the right atrial appendage as the first implant location and the extension body may be located adjacent the left ventricle as the second implant location, with the controller delivering dual chamber sensing and pacing.

Optionally, the IIMD may be anchored in the ventricular vestibule such that the first activation site is within the conductive network of a right ventricle and the extension body is located adjacent the left ventricle as the second implant location, with the controller delivering dual chamber sensing and pacing.

In accordance with the embodiment, a method is provided for implanting an intra-cardiac system. The method comprises maneuvering an introducer assembly into a local chamber of a heart; pushing an IIMD out of a sheath of the introducer assembly toward a first implant location; anchoring the IIMD to the first implant location with a first electrode located at a first activation site within a conductive network of a first chamber; moving the sheath away from the IIMD; maneuvering the introducer assembly into a coronary sinus toward a vessel of interest; discharging an IC device extension out of the sheath at a second implant location such that a second electrode on the IC device extension located at a second activation site in the vessel of interest proximate to a second chamber of the heart; and configuring a controller, within the IIMD, to cause stimulus pulses to be delivered through at least one of the first and second electrodes to at least one of the local and distal activation sites, respectively.

The anchoring operation may locate the IIMD in a right atrium as the local chamber with the first activation site at one of the right atrial appendage and ventricular vestibule. The discharging operation may position the IC device extension in a lateral coronary vein as the vessel of interest with the second activation site proximate to a left ventricle as the second chamber. The anchoring operation may locate the IIMD in a right ventricle with the first activation site in the right ventricle as the first chamber; and wherein the discharging operation positions the IC device extension such that the second activation site is proximate to a left ventricle as the second chamber.

Optionally, the anchoring operation locates the IIMD in a right atrium with the first activation site in the right atrium as the first chamber, and the discharging operation positions the IC device extension such that the second activation site is proximate to a left atrium as the second chamber. Optionally, the introducer assembly includes a pusher removably secured to the IIMD and a placement tool extending into a lumen in the IC device extension. The pusher manipulates and anchors the IIMD at the first implant location. The placement tool locates the IC device extension at the second implant location.

Optionally, the placement tool represents one of a stylet and a guide wire. The discharging operation comprises using a stylet within the sheath to maneuver the second electrode into the second activation site. The method further comprises pre-forming the extension body with an active segment in a curved shape having a trough, the second electrode located in the trough, the curved shape configured to following a contour of an interior of the vessel of interest.

Optionally, the method further comprises loading the IC device extension into the sheath such that a memorized, pre-formed non-linear shape of the IC device extension is changed to a temporary, extended or dilated introducer state; and retracting the introducer assembly such that, as the IC device extension is discharged from a distal end of the sheath, the IC device extension returns to the memorized, pre-formed non-linear shape.

The method may further comprise, forming the device extension with a stabilizer segment, and permitting the stabilizer segment to bend into a curved shape sufficient to extend into and engage a contour of an interior of the vessel of interest.

### BRIEF DESCRIPTION OF THE DRAWINGS

FIG. 1 illustrates a sectional view of the patient's heart with an intra-cardiac implantable medical device and intra-cardiac device extension implanted in accordance with an embodiment of the present invention.

FIG. 2 illustrates a side view of an introducer assembly, according to an embodiment.

FIG. 3A illustrates a top plan view of the sheath of FIG. 2.

FIG. 3B illustrates an end plan view of a sheath formed in accordance with an alternative embodiment.

FIG. 3C illustrates a distal plan view of a sheath formed in accordance with an embodiment.

FIG. 4 illustrates an extension body and placement tool according to an embodiment.

FIG. 5 illustrates an extension body and placement tool according to an embodiment.

FIG. 6 illustrates a distal end of an extension body formed in accordance with an alternative embodiment.

FIG. 7 illustrates an initial implant step in an exemplary process for implanting an IIMD in accordance with an embodiment.

FIG. 8 illustrates an intermediate implant step in an exemplary process for implanting an IIMD in accordance with an embodiment.

FIG. 9 illustrates an enlarged view of a portion of the coronary sinus and vessels joined to the coronary sinus with an IC device extension deployed according to an embodiment.

FIG. 10 illustrates a portion of the extension body located in the CS proximate to the LA when deployed in accordance with an embodiment.

FIG. 11 shows a block diagram of an IIMD in accordance with an embodiment.

FIG. 12A illustrates an IIMD formed in accordance with an alternative embodiment.

FIG. 12B illustrates the IIMD once the sheath has been removed and the placement tool has been withdrawn.

FIG. 13 illustrates an IIMD and stabilizer segment formed in accordance with an alternative embodiment.

FIG. 14 illustrates an IIMD system formed in accordance with an alternative embodiment.

### DETAILED DESCRIPTION

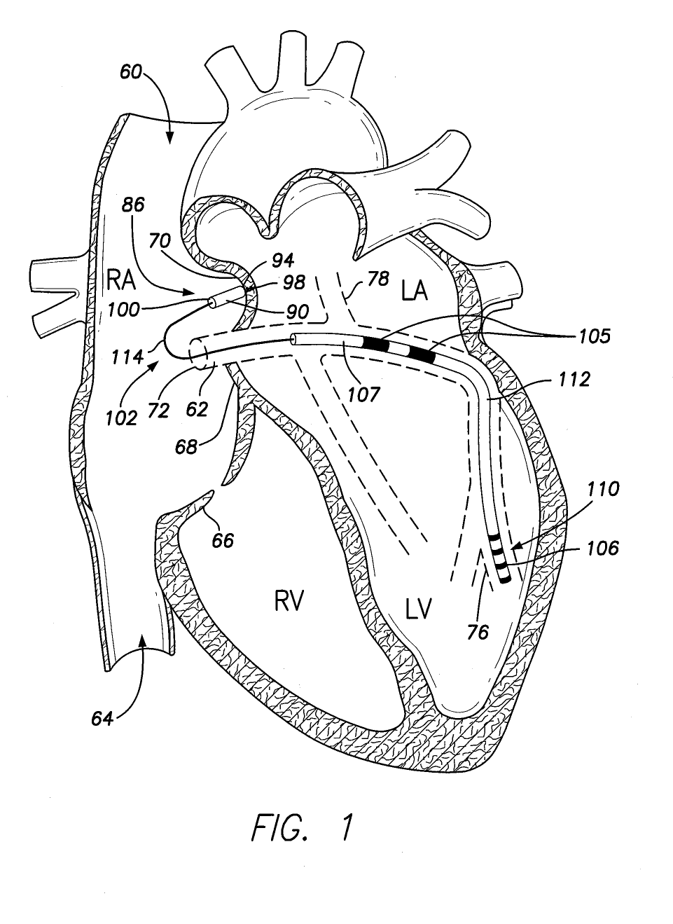

FIG. 1 provides a sectional view of the patient's heart, showing the right and left atrium (RA and LA), and right and left ventricles (RV and LV), with an intra-cardiac implantable medical device (IIMD) 86 and intra-cardiac (IC) device extension 102 (also referred to as an ICDE) implanted in accordance with an embodiment of the present invention. The IIMD 86 may have been placed through the superior vena cava (SVC) or inferior vena cava (IVC) into the right atrium of the heart. As shown in FIG. 1, the right atrium wall includes the superior vena cava inlet 60, coronary sinus 62, IVC inlet 64, tricuspid valve 66, and the ventricular vestibule (VV) region 68. The ostium (OS) 72 illustrates the juncture of the coronary sinus 62 and the RA. The coronary sinus branches into various tributary vessels such as the lateral veins, great cardiac vein, middle cardiac vein, small cardiac vein, anterior inter-ventricular veins and the like. In FIG. 1, the lateral cardiac vein 76 and vein of Marshall 78 are denoted with reference numbers as examples. The lateral cardiac vein 76 extends along the LV toward the LV apex. The vein of Marshall 78 extends along a side of the LA.

The IIMD 86 may be implanted in various locations within a “local chamber” of the heart, such as the RA, RV, LA and LV and at various activation sites of interest. The term “local chamber” shall be used to describe the chamber in which the IIMD 86 is physically implanted. The term “adjacent chamber” shall be used to describe one or more of the chambers other than the local chamber. For example, the IIMD 86 may be implanted in the RA as the local chamber and at an activation site of interest that is in the right atrial appendage (RAA) region 70 to sense/stimulate the right atrium. The term “activation site” shall be used to describe the tissue location where a sense and/or pace electrode is located and associated with the conduction network of a chamber of interest. The activation sites may or may not correspond to the conductive network of the local chamber where the IIMD 86 is physically located. The RAA region 70 represents a first activation site that is associated with the chamber in which the IIMD 86 is implanted, namely the local (RA) chamber, given that contractions may be initiated in the RA when stimulus pulses are delivered to the surface tissue in the RAA region 70. Optionally, the IIMD 86 may be implanted in the RA as the local chamber, but at an activation site of interest in the ventricular vestibule 68 located adjacent to the tricuspid valve 66 along a segment of the tricuspid annulus. The VV region 68 constitutes a first activation site that is not associated with the chamber in which the IIMD 86 is implanted (the RA), given that contractions may be initiated in the right ventricle when stimulus pulses are delivered in the VV region 68.

The IIMD 86 may be operated in various modes, such as in select pacemaker modes, select cardiac resynchronization therapy modes, a cardioversion mode, a defibrillation mode and the like. For example, a typical pacing mode may include DDIR, R, DDOR and the like, where the first letter indicates the chamber(s) paced (e.g., A: Atrial pacing; V: Ventricular pacing; and D: Dual-chamber (atrial and ventricular) pacing). The second letter indicates the chamber in which electrical activity is sensed (e.g., A, V, or D). The code O is used when pacemaker discharge is not dependent on sensing electrical activity. The third letter refers to the response to a sensed electric signal (e.g., T: Triggering of pacing function; I: Inhibition of pacing function; D: Dual response (i.e., any spontaneous atrial and ventricular activity will inhibit atrial and ventricular pacing and lone atrial activity will trigger a paced ventricular response) and 0: No response to an underlying electric signal (usually related to the absence of associated sensing function)). The fourth letter indicates rate responsive if R is present. As one example, the IIMD 86 may be configured with DDI, DDO, DDD or DDDR mode-capability when placed at a local activation site in the RA.

The IIMD 86 comprises a housing 90 configured to be implanted entirely within a single local chamber of the heart. The housing 90 includes a proximal base end 94 and a distal top end 100. The proximal base end 94 includes an active fixation member 98, such as a helix, that is illustrated to be implanted in the RAA region 70. A shaped IC device extension 102 extends from the distal top end 100 of the housing 90. The IC device extension 102 may be tubular in shape and may include a metal braid provided along at least a portion of the length therein. The IC device extension 102 includes a transition segment 114 and one or more active segment(s) 110. Optionally, the IC device extension 102 may include one or more stabilizer segment(s) 112 as well. The active and stabilizer segments 110 and 112 may be interspersed in various combinations, that collectively device an elongated body 107.

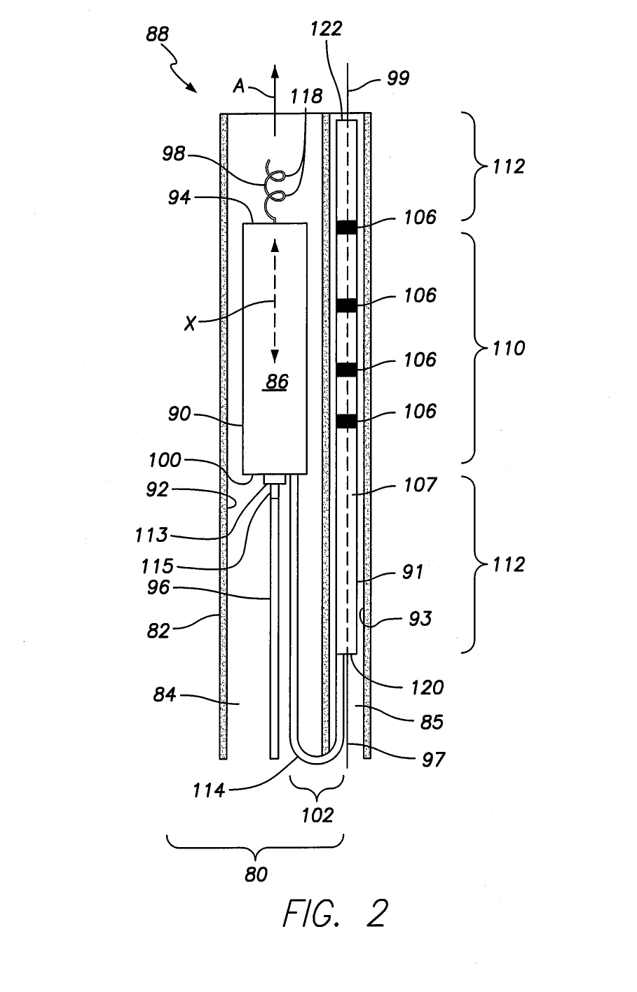

As explained herein, during implantation, the IC device extension 102 is held in an elongated, straight shape within a sheath 82 (FIG. 2). After implanted, once the sheath 82 is removed, when in a deployed configuration, the IC device extension 102 returns to an initial pre-formed state and shape. For example, the active, stabilizer, and/or transition segments 110-114 of the IC device extension 102 may be formed straight and thus, when implanted simply lay within the vein or vessel. Alternatively, the active, stabilizer, and/or transition segments 110-114 may be formed in a curved non-linear state such that, when deployed from the sheath 82, the active and/or stabilizer segments 110 and 112 bend, curve and/or coil until becoming preloaded against anatomical portions of tissue of interest within the vein or vessel in which the IC device extension 102 is implanted, while the transition segment 114 bends toward the OS. The stabilizer segment(s) 112 curve to firmly, passively engage walls of the vein or vessel to hold the IC device extension 102 in a fixed location. The stabilizer segment(s) 112 may be located on opposite sides of the active segment 110.

Optionally, the stabilizer segment 112 may be located distally beyond an outermost electrode 106 in the active segment 110. Optionally, the stabilizer segment 112 may be located proximally near the transition segment 114 before an inner electrode 105 in the active segment 110. Optionally, the stabilizer segment(s) 112 may be omitted entirely.

The IC device extension 102 is formed with shape memory characteristics that allow the IC device extension 102 to transform between a collapsed state, in which the IC device extension 102 assumes a substantially linear shape, and an expanded state, in which the IC device extension 102 assumes a multi-curved shape. In one embodiment and depending on the vessel designed for implant, the curved configuration of the IC device extension 102 may comprise multiple tightly curved segments, obtusely curved segments, generally linear regions and the like. The number, length, and order of the segments and regions, as well as the degree to which individual segments or regions are curved or linear may vary depending upon the anatomical contour to be followed. The shaped IC device extension 102 is formed into a pre-loaded shape in which various regions or segments extend along desired arcuate paths and project from longitudinal/lateral axes at desired pitch, roll and yaw angles, where the pitch, roll and yaw angles are measured from reference angular positions.

One or more electrodes 106 are located along the active segment 110 that is proximate to the LV apex. Optionally, the electrode(s) 105 may be provided in a second active segment 110 proximate to the LA. Optionally, the electrodes 105 or 106 may be omitted entirely.

FIG. 2 illustrates a longitudinal side view of an introducer assembly 80, according to an embodiment. The introducer assembly 80 includes a flexible, longitudinal, cylindrical open-ended sheath 82 defining at least a central internal passage 84. The sheath 82 includes an open distal end 88. The sheath 82 may be a flexible tube formed of silicon rubber, for example, that is configured to be maneuvered through patient anatomy, such as veins and the heart. In this respect, the sheath 82 may be similar to that of a cardiac catheter. Optionally, introducer assembly 80 may include one or more peripheral passages 85 extending parallel to, and along a side of, the central internal passage 84.

Optionally, the sheath 82 may have a single internal passage 84, without any peripheral passages. The ICDE 102 may be located adjacent or behind the IIMD 86 in the passage 84. For example, the ICDE 102 may be configured into one or more loops in the area adjacent the pusher rod 96 with the extension body 107 located behind the IIMD 86 and extending along a side of the pusher rod 96. The transition segment 114 could extend rearward along the passage 84, thereby and permitting overall outer diameter of the sheath 82 to be only slightly larger than the outer diameter of the housing 90 of the IIMD 86.

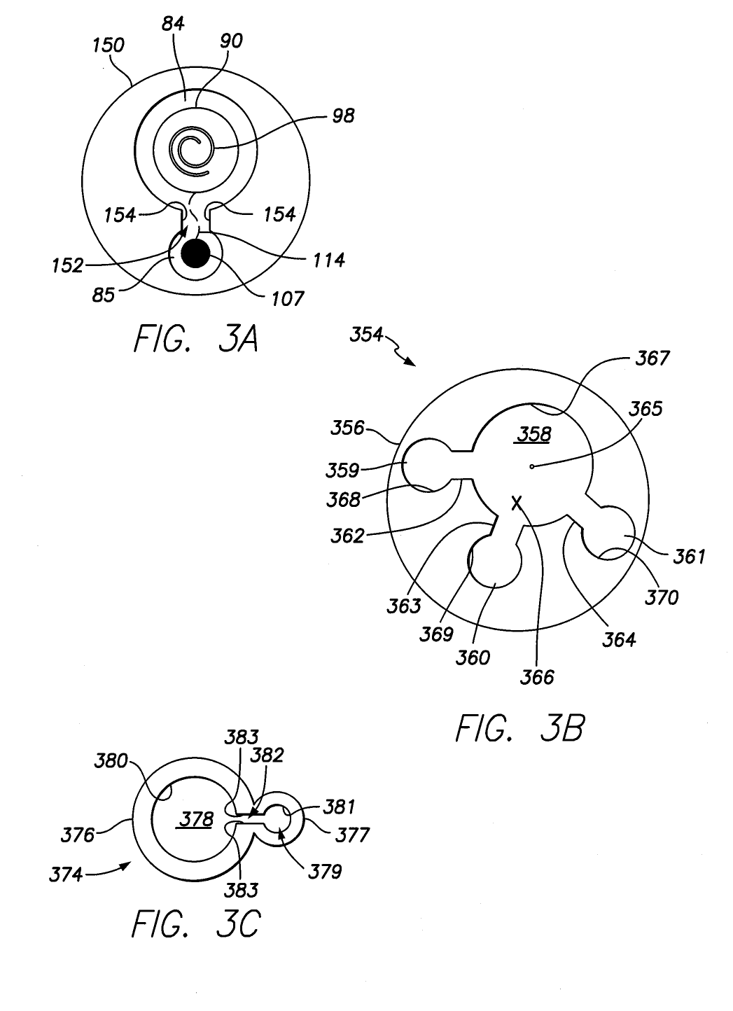

FIG. 3A illustrates a top plan view of the sheath 82 of FIG. 2. The sheath 82 has an outer envelope with an even circular contour to form a main outer wall 150. The primary central passage 84 is provided within the main outer wall 150. Optionally, at least one secondary peripheral passage 85 may be provided. The housing 90 and active fixation member 98 of the IIMD 86 are illustrated to be positioned within the passage 84, while the extension body 107 (comprising the active and stabilizing segments) is illustrated to be positioned within the passage 85. The transition segment 114 electrically and physically couples the IIMD 86 and IC device extension 102. The transition segment 114 of the IC device extension 102 is shown in dashed line passing through the passage linking slot 152. The passages 84 and 85 have corresponding smooth inner walls 92 and 93, respectively. The passages 84 and 85 are joined and communicate with one another through a linking slot 152. The slot 152 has opposed facing sides 154. The slot 152 extends along the length of the sheath 82 to permit movement of the transition segment 114 between the passages 84 and 85 during deployment.

Optionally, more than one ancillary passage 85 may be provided about the passage 84. Optionally, the passages 84 and 85 may be symmetrically or evenly distributed about a center axis of the sheath 82. The passages 84 and 85 are directly exposed to one another through the passage linking slot 152 that extends along at least a portion of the length of the passages 84 and 85. The slot 152 also opens on to the distal end 88 of the sheath 82.

When the IIMD 86 and IC device extension 102 are loaded (either through the distal or proximal ends) into the sheath 82, the transition segment 114 traverses the slot 152. The transition segment 114 travels longitudinally along the slot 152 during implantation and is entirely discharged from the slot 152 at the distal end 88 once the IIMD 86 and IC device extension 102 are fully deployed and engaged to tissue of interest.

FIG. 3B illustrates an end plan view of a sheath 354 formed in accordance with an alternative embodiment. In FIG. 3B, the sheath 354 has an outer wall with an outer envelope 356 that has a continuous circular cross-section. The sheath 354 also includes multiple passages 358-361. For example, a primary passage 358 may be circular or oval with a larger cross sectional area than the cross-section of secondary passages 359-361. The passages 359-361 are connected to the passage 358 through linking slots 362-364, respectively. Optionally, the passages 359-361 may be connected to one another through linking slots (not shown). The passages 358-361 have various cross-sectional shapes, such as circular, oval, square, rectangular, triangular, hexagonal, polygonal and the like. The passages 359-361 are located along one arcuate circumferential portion of the passage 358. The passage 358 is located with the center 365 offset from a center 366 of the sheath 354. Centers of the passages 359-361 are radially displaced from the center 366 of the sheath 354. The passages 359-361 may have common or different diameters, cross-sectional shapes, spacing from the passage 358, and spacing between one another. Optionally, the passages 359-361 may be grouped closer to one another, or evenly distributed about the circumference of the passage 358. The passages 358-361 have smooth interior walls 367-370.

FIG. 3C illustrates a distal plan view of a sheath 374 formed in accordance with an embodiment. The sheath 374 has an outer envelope with an uneven contour to form a main outer wall 376 and an ancillary wall segment 377. The ancillary wall segment 377 is located along one side of the main outer wall 376. A primary passage 378 is provided within the main outer wall 376, while at least one secondary passage 379 is provided within the ancillary wall segment 377. The passages 378 and 379 have corresponding smooth inner walls 380 and 381, respectively. The passages 378 and 379 are joined and communicate with one another through a linking slot 382. The slot 382 has opposed facing sides 383 that extend along the length of the sheath 374. Optionally, more than one ancillary wall segment 377 and passage 379 may be provided about the passage 378.

Returning to FIG. 2, a physician or surgeon operates the introducer assembly 80 at a proximal end (not shown). The proximal end may include controls that allow the sheath 82 to be bent, curved, canted, rotated, twisted, or the like, so as to be navigated through a patient's vasculature and maneuver the distal end 88 to first implant location within a chamber of interest, representing the local chamber. In an embodiment, the distal end 88 of the sheath 82 may be bent, curved, canted, rotated, twisted, articulated, or the like through operation by the physician or surgeon manipulating the proximal end of the assembly 80.

As shown in FIG. 2, the IIMD 86 and IC device extension 102 are loaded into passages 84 and 85 of the sheath 82 and held within the sheath 82 at the distal end 88. The outer wall of the housing 90 of the IIMD 86 slides along inner wall 92 of the central internal passage 84 of the sheath 82, while the outer wall 91 of the IC device extension 102 slides along the inner wall 93 of the peripheral passage 85. The IIMD 86 and IC device extension 102 are configured to be pushed out of, or ejected from, the sheath 82 in the direction of arrow A. The distal top end 100 of the IIMD 86 connects to a pusher rod 96 extending within the sheath 82. For example, the distal top end 100 may be connected to the pusher rod 96 through a threadable connection, an interference fit, or the like. The pusher rod 96 may be aligned generally coaxial with the IIMD 86. An active fixation member 98, such as a helical anchor extends from a distal end 100 of the IIMD 86. The helical anchor may be a coiled, helical wire having a sharp point at a distal end. While the active fixation member 98 is shown as a helical anchor, the active fixation member 98 may alternatively be a hook, barb, or the like, that is configured to secure the IIMD 86 into tissue of the heart wall. The active fixation member 98 may include one or more electrodes 118 for pacing and/or sensing.

The transition segment 114 of the IC device extension 102 represents a non-lead wire segment that electrically couples the IIMD 86 to one or more electrodes 106. The transition segment 114 of the IC device extension 102 has a “non-lead” structure in that remote manipulation of the IC device extension 102 is not sufficient to locate the electrode 106 at a desired position. As shown in FIG. 2, the active, stabilization and transition segments 110, 112 and 114 are straightened when in passage 85. FIG. 2 illustrates the IC device extension 102 in more detail with the transition segment 114 electrically and physically connected at one end to the distal top end 100 of the housing 90 of the IIMD 86 and at another end to a proximal end 120 of the extension body 107 that includes the active and stabilization segments 110 and 112. The active segments 110 carry one or more electrode(s) 105, 106. In the configuration shown in FIG. 2, there is slack in the transition segment 114. The IC device extension 102 includes one or more conductors within an insulated sheath. Multiple conductors may be braided together as a single electrical path or may be insulated from one another to provide a desired number of distinct electrical paths to/from the IIMD 86 and one or more electrodes 106. Optionally, a plurality of electrically separate wires 102 may be utilized when an equal plurality of electrodes 102 are provided.

The extension body 107 includes a proximal end 120 and a distal end 122 with a lumen extending there between. The lumen within the extension body 107 is open at least at the proximal end 120. The extension body 107 receives an ICDE placement tool 97, such as a guide wire, pusher rod, stylet and the like, through the proximal end 120 into the lumen. The ICDE placement tool 97 may include a combination of components, such as a guide wire and pusher rod.

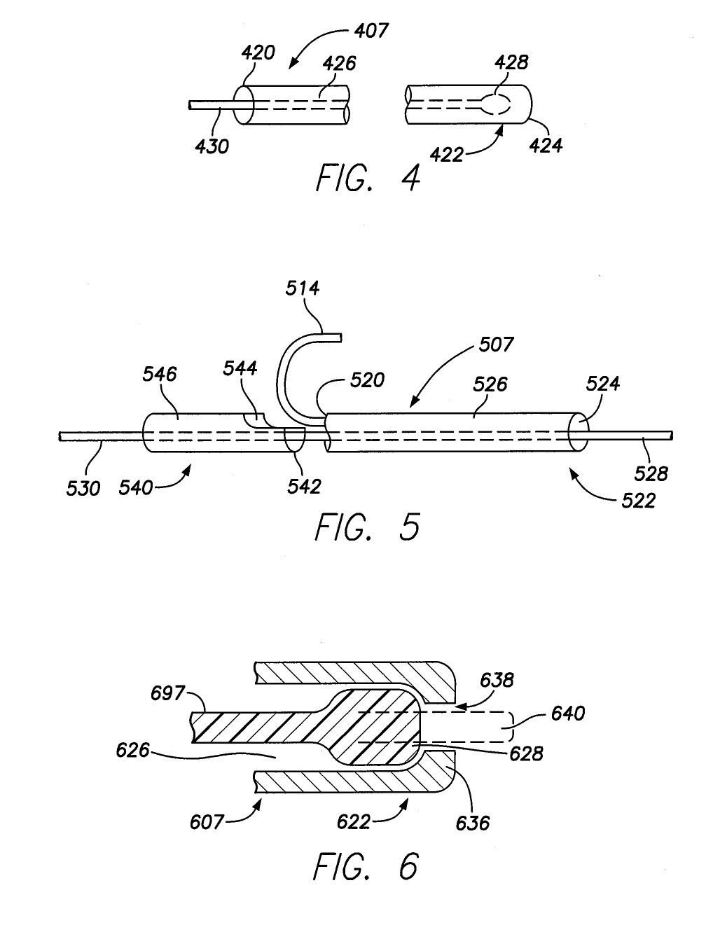

FIGS. 4-6 illustrate alternative configurations for distal ends for the extension body of the IC device extension 102. FIG. 4 illustrates an extension body 407 that includes a proximal end 420, a distal end 422 and a lumen 426 that extends there between. The distal end 422 is closed by a termination wall 424. A stylet 430 forms the ICDE placement tool 97 and has an outer termination end 428 that is enlarged and rounded to abut against the termination wall 424. During implantation, the stylet 430 pushes against the termination wall 424 to advance and maneuver the extension body 407 to a desired implant location and activation site. Once the extension body 407 is in the desired location, the stylet 430 is withdrawn along the lumen 430.

FIG. 5 illustrates an extension body 507 that includes a proximal end 520, a distal end 522 and a lumen 526 that extends there between. The distal end 522 has an opening 524 there through. A guide wire 530 and an ICDE pusher rod 540 collectively form the ICDE placement tool 97. The guide wire 530 has an outer termination end 528 that is rounded but extends through the opening 524 at the distal end 522 of the extension body 507. A transition segment 514 electrically and physically couples the extension body 507 to an IIMD (not shown). During implantation, the guide wire 530 is advanced to a desired position within a vein designated for implant of the IC device extension (ICDE implant vein). The extension body 507 is then advanced along the guide wire 530 to a desired ICDE implant location. Once the extension body 507 is in the desired location, the guide wire 530 is withdrawn along the lumen 530.

The pusher rod 540 is slidably loaded over the guide wire 530. Only a distal portion of the pusher rod 540 is illustrated. The pusher rod 540 includes a pusher lumen 546 extending along a length thereof and configured to slidably receive the guide wire 530. A distal end 542 of the pusher rod 540 abuts against the proximal end 520 of the extension body 507 when the pusher rod 540 is advanced and used to urge/push the extension body 507 along the ICDE implant vein to the ICDE implant location.

The pusher rod 540 includes a notch 544 extending rearward from the distal end 542. The notch 544 defines an opening that receives the transition segment 514. The notch 544 prevents the transition segment 514 from interfering with mating engagement between the distal end 542 of the ICDE pusher rod 540 and the proximal end 520 of the extension body 507.

After the ICDE pusher rod 540 completes the procedure of advancing the extension body 507 to the ICDE implant location, next the guide wire 530 is removed/withdrawn. If needed, the ICDE pusher rod 540 may remain in contact with the extension body 507 to prevent shifting (e.g., partial withdraw) of the extension body 507 as the guide wire is removed. Next, the ICDE pusher rod 540 is removed/withdrawn.

Optionally, the distal end 542 and proximal end 520 may include corresponding mating features that allow a temporary secure connection therebetween. The distal and proximal ends 542 and 520 may be secured to one another during ICDE implant and then disconnected when the ICDE pusher rod 540 is removed. Optionally, the guide wire 530 may be omitted entirely or only used to the extend desired to guide the distal end 528 of the extension body 507 into the coronary sinus and/or a select tributary vein.

FIG. 6 illustrates an extension body 607 having a distal end 622 formed in accordance with an alternative embodiment. The extension body 607 includes a lumen 626 that ends at the distal end 622. The distal end 622 includes a flange 636 that partially closes the end of the lumen 626. The lumen 626 is configured to receive a placement tool 697. In the example of FIG. 6, an end of the placement tool 697 is shown in solid lines as a stylet with an enlarged, rounded end 628 shaped and dimensioned to fit against the flange 636, thereby preventing the stylet from exiting the distal end 622 of the extension body 607.

The flange 636 includes a guide wire passage 638 that is configured to permit a guide wire (denoted in dashed lines 630) to pass there through. In the example of FIG. 6, the end of the placement tool 697 is also shown in dashed lines as a guide wire with a smaller distal end 640 that is dimensioned and shaped to pass through the passage 638 in the flange 636, thereby permitting the guide wire to extend beyond the distal end 622 of the extension body 607. It should be understood that FIG. 6 illustrates alternative ends for the placement tool, one alternative in solid lines while the other alternative is shown in dashed lines.

Returning to FIG. 2, the pusher rod 96 includes a coupling member 115, for example, a threaded region, at a distal end that connects to the tool receptacle 113. As shown in FIG. 2, the pusher rod 96 extends from the IIMD 86 about a central axis X. As such, the pusher rod 96 is aligned generally coaxial with the IIMD 86. The sheath 82 and pusher rod 96 are configured to guide the IIMD 86 to a desired portion of heart wall tissue. The distal end of the pusher rod 96 fits into the IIMD 86 through a threaded connection, a friction fit, a snap fit, or the like. The pusher rod 96 is configured to be removed from the IIMD 86 once the IIMD 86 is anchored into the atrial wall. That is, the strength of the connection between the distal end of the pusher rod 96 and the tool receptacle 113 may be overcome by a pulling force on the pusher rod 96 once the IIMD 86 is anchored into the atrial wall.

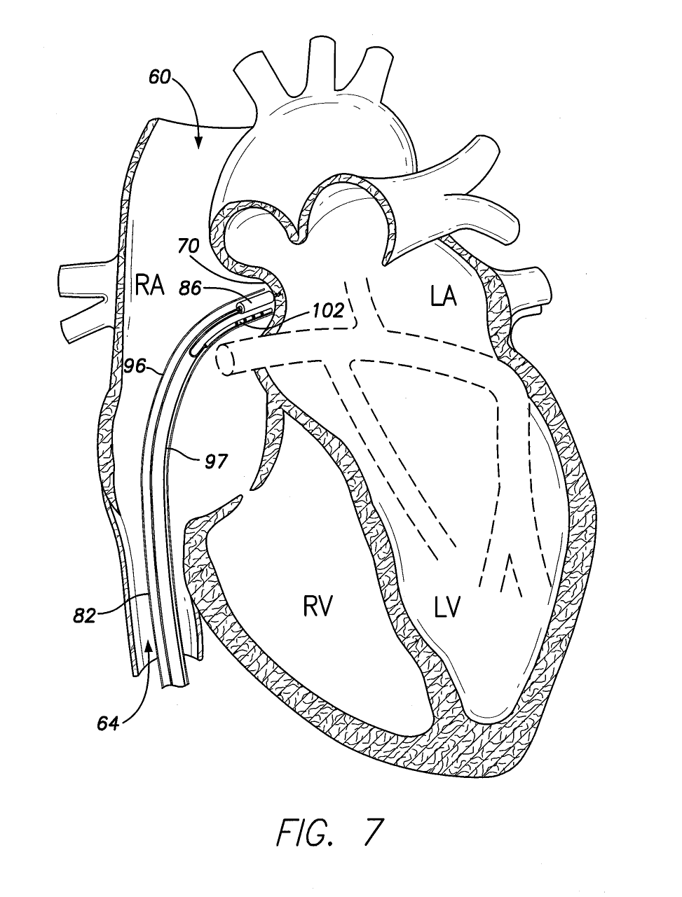

Next, an exemplary implantation process will be explained in connection with FIGS. 7-9. In operation, the introducer assembly 80 is inserted into a vein of a patient and maneuvered toward the patient's heart. In particular, a physician maneuvers the introducer assembly 80 through human vasculature, such as veins, and into the heart, by way of the superior vena cava 60 or the interior vena cava 64. During this time, a separate and distinct imaging system, such as a fluoroscopic imaging system, and/or a surgical navigation system may be used to assist in guiding the introducer assembly 80 into the heart. For example, a physician may view a real-time fluoroscopic image of the patient's anatomy to see the introducer assembly 80 being maneuvered through patient anatomy.

FIG. 7 illustrates an initial implant stage or step in an exemplary process for implanting an IIMD in accordance with an embodiment. The introducer assembly 80 is maneuvered and introduced through the IVC 64 into the heart and into the right atrium. The introducer assembly 80 is then manipulated until the distal end 88 thereof is located proximate to a first implant location, such as the RAA, the VV, the apex of the RV and the like. In the example of FIG. 7, the introducer assembly 80 is then manipulated until the distal end 88 thereof is located proximate to the RAA region 70. Once the distal end 88 of the sheath 82 contacts the tissue at the implant site, the pusher rod 96 is pushed toward the tissue until the active fixation member 98 engages the tissue of interest. During this time, the pusher rod 96 is also rotated about the axis X, thereby causing the IIMD 86 and the active fixation member 98 to rotate in a common direction. As such, the active fixation member 98 is screwed into the tissue of the heart wall and the IIMD 86 is anchored into the tissue of interest.

Optionally, the introducer assembly 80 may be inserted through the SVC. Optionally, when it is desirable to locate the IIMD 86 in the RV, once entering the RA, the introducer assembly 80 manipulated to pass through the tricuspid valve 62 and into the right ventricle. The introducer assembly 80 is then maneuvered toward the right ventricular apex until the distal end 88 of the sheath 82 is proximate or abuts against tissue of interest. The pusher rod 96 is rotated to actively affix the IIMD 86 to the RV apex.

In embodiments described herein, the IIMD 86 and/or IC device extension 102 are able to rotate within and relative to the sheath 82. Optionally, the sheath 82 may include one or more anti-rotation keying features along at least one area on the inner wall 92, 93. For example, a bump or other raised projection may be formed to extend inward from the inner wall 92 and/or 93 and oriented to direct toward the IIMD 86 and/or IC device extension 102. For example, when the projection is provided on a post or other member projecting inward from the inner wall 92, the mating indent or notch may be provided along the outside of the IIMD 86. The projection and notch engage one another to prevent internal rotation of the IIMD 86 within the sheath 82 while engaged.

Optionally, instead of the active fixation member 98, a barb may extend from the proximal end 94 of the IIMD 86. In this embodiment, the IIMD 86 may simply be pushed into the heart wall in order to anchor the IIMD 86 thereto, instead of also rotated. Once the IIMD 86 is anchored to the heart wall, the pusher rod 96 is pulled back in the direction opposite to arrow A. As the pusher rod 96 is pulled back, the anchoring force of the active fixation member 98 (or barb) ensures that the IIMD 86 remains anchored to the heart wall. The anchoring force ensures that the pusher rod 96 separates from the IIMD 86 (as the pusher rod 96 may only be connected to the IIMD 86 through a relatively weak interference fit, for example).

After the pusher rod 96 separates from the IIMD 86, the sheath 82 is also pulled back in the direction opposite to arrow A (FIG. 2). Because the IIMD 86 is now anchored to the heart wall, the IIMD 86 slides out of engagement with the sheath 82. During this time, the transition segment 114 of the IC device extension 102 is fed along the slot 152 from the open distal end 88 as the sheath 82 continues to pull away from the IIMD 86, while the extension body 107 is held within the passage 85.

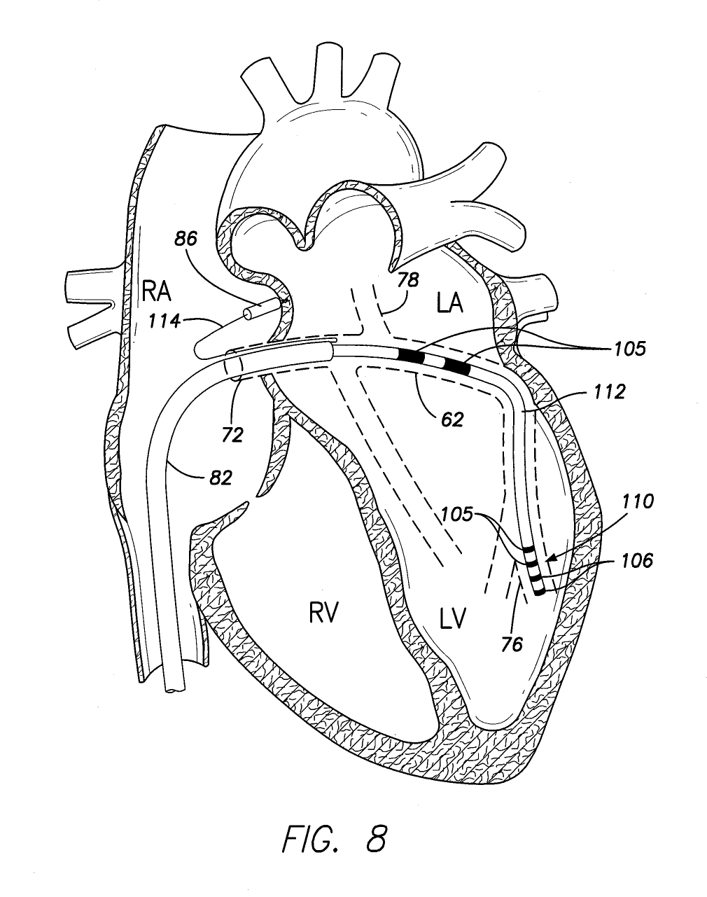

FIG. 8 illustrates an intermediate implant stage or step in an exemplary process for implanting an IIMD in accordance with an embodiment. Once the sheath 82 is withdrawn from the IIMD 86, the sheath 82 is maneuvered such that at least the distal end 88 enters the ostrium 72 and progresses a predetermined distance into the coronary sinus 62. The sheath 82 may be inserted a short or long distance into the CS 62. For example, the sheath 82 may be advanced until the distal end 88 is located within an implant vein of interest (e.g., into the lateral cardiac vein 76). Alternatively, the distal end 88 of the sheath 82 may be only slightly introduced into an initial portion of the CS 62 and then stopped.

Once the sheath 82 is advanced the desired distance into the CS 62, next the ICDE placement tool 97 is controlled to advance the IC device extension 102 to the desired implant location in the vessel of interest. The vessel of interest may be any one of various vessels, such as the great cardiac vein, middle cardiac vein, lateral cardiac vein and the like. For example, when the ICDE placement tool 97 is a stylet 430, the stylet 430 has an enlarged, rounded end 428 that pushes against a closed termination end 424 of the distal end 422 of the extension body 407 to advance the IC device extension 102 to the desired implant location. In one embodiment, the stylet 430 also maintains the IC device extension 102 in a relatively straight configuration and guides the IC device extension 102 along the CS 62 and lateral cardiac vein 76 until the electrodes 106 are located proximate to the apex of the LV. Once the electrodes 106 are located at the LV apex, the stylet 430 is withdrawn from the lumen 426 in the extension body 407. As the stylet 430 is withdrawn, the extension body 407 is permitted to return a natural pre-formed shape, thereby permitting any stabilization segments 112 therein to curve and bend to a stabilizing shape.

As another example, when the ICDE placement tool 97 is a guide wire 530, the guide wire 530 extends through the opening 524 at the distal end 522 of the extension body 507. The guide wire 530 is advances to the desired implant location. Once the guide wire 530 is located at the desired implant location in the implant vein of interest, next the IC device extension 102 is advanced over the guide wire 530 until the electrodes 106 are located proximate to the apex of the LV (as one example). The guide wire 530 maintains the elongated body 507 in a relatively straight configuration and guides the extension body 507 along the CS 62 and lateral cardiac vein 76. Once the electrodes 106 are located at the LV apex or other implant location, the guide wire 530 is withdrawn from the lumen 526 in the extension body 507. As the guide wire 530 is withdrawn, the extension body 507 is permitted to return a natural pre-formed shape, thereby permitting any stabilization segments 112 therein to curve and bend to a stabilizing shape. The sheath 82 and ICDE placement tool 97 are then removed from the heart.

Optionally, the operations of the implant process described in connection with FIGS. 7 and 8, may be performed in either order. For example, the IIMD may be maneuvered into the local chamber, then pushed to (and anchored at) the first implant location before or after the ICDE is maneuvered into the CS and discharged at the second implant location. Thus, the ICDE may be implanted first, followed by implant of the IIMD, or vice versa.

Optionally, when the IIMD 86 and/or IC device extension 102 are loaded into the sheath 82, the transition segment 114 may be pre-wound by a desired number of turns around the pusher rod 96 and/or placement tool 97, respectively. The transition segment 114 is pre-wound in a reverse direction opposite to the direction in which the active fixation member 98 is turned. For example, when it is desirable to pre-wind the transition segment 114 about the IIMD 86 and if the active fixation member 98 is expected to use 1-10 clockwise turns to screw in a helix, then the transition segment 114 may be pre-wound in an equal number of 1-10 turns in the counterclockwise direction about the pusher rod 95.

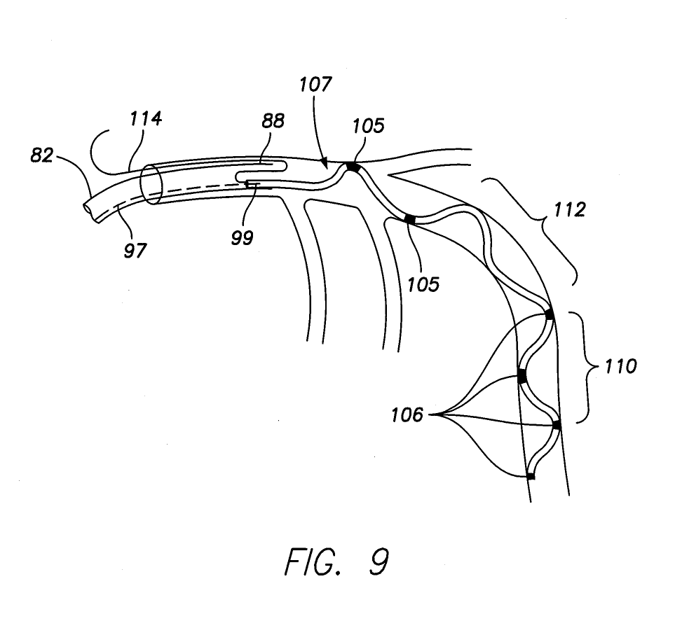

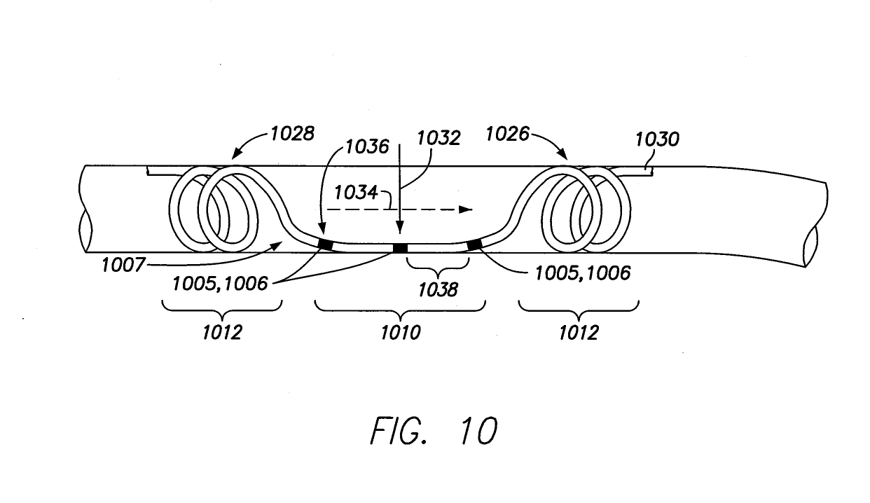

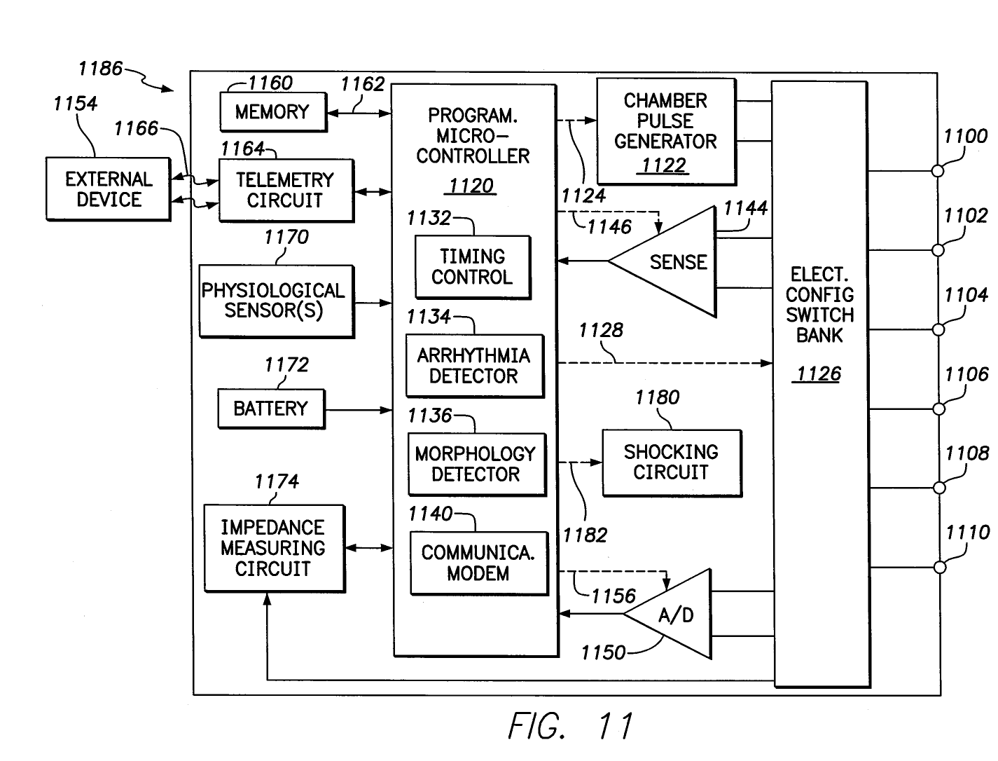

FIG. 9 illustrates an enlarged view of a portion of the coronary sinus and various veins joined to the coronary sinus. The distal end 88 of the sheath 82 is illustrated with the transition segment 114 wrapping over the distal end 88 toward the IIMD (not shown). The ICDE placement tool 97 is partially withdrawn from the elongated body 107, thereby permitting the active and stabilization segments 110, 112 to return to their natural pre-formed shapes. The curves and bends in the active and stabilization segments 110, 112 traverse the cross section of a corresponding vessel of interest multiple times to engage tissue along the vessel at various points. The electrodes 105 are located to engage tissue along the LA, while the electrodes 106 are located to engage tissue along the LV. The stabilizing shape formed by the active and stabilization segments 110, 112 prevents the elongated body 107 from moving an unduly large distance along the length of the vessel.

The extension body 107 is formed with an outer layer made of a biocompatible insulated material such as EFTE, silicon, OPTIM and the like. Internal structures of the exemplary embodiments of the extension body 107 are discussed below. In general, the extension body 107 is formed of materials that are flexible yet exhibit a desired degree of shape memory such that once implanted, the active segment 110 and stabilizer segment 112 are biased to return to a pre-formed shape. One or more insulated conductive wires are held within the extension body 107 and span from the IIMD 86 to any sensors or electrodes provided on the extension body 107.

One or more stabilizer segments 112 may be located at intermediate points and/or the distal end of the extension body 107 and in one or more pre-formed shapes that are biased to extend slightly outward in a lateral direction relative to a length of the extension body 107. The stabilizer segment 112 engages a first region of the vein wall or tissue. For example, the stabilizer segment 112 may extend upward into and engage a vein wall against the LA and/or against the LV.

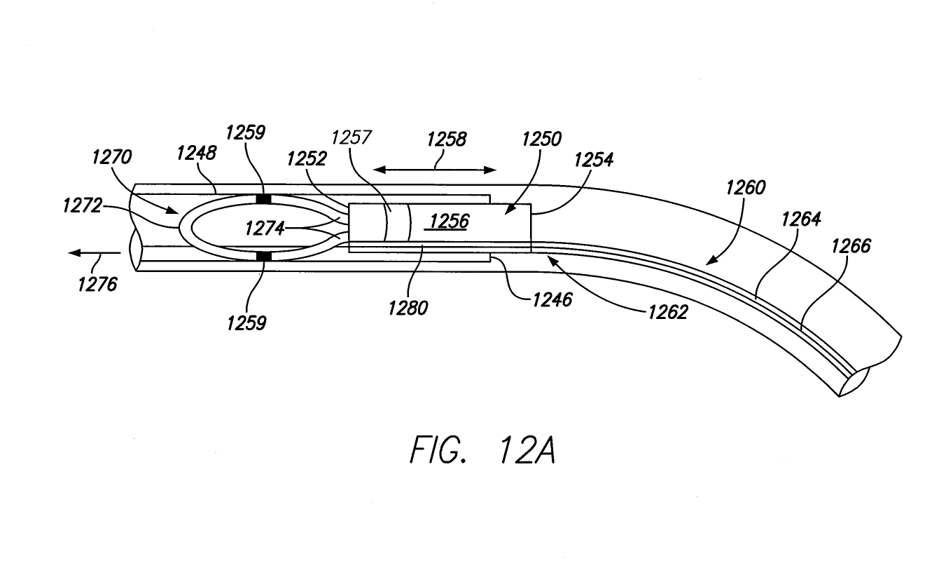

FIG. 10 illustrates a portion of an extension body 1007 formed in accordance with an embodiment. The extension body 1007 is located in the CS proximate to the LA when deployed in accordance with an embodiment. The stabilizer segments 1012 are pre-formed into a predetermined shape based upon which portion of the CS and tributaries are to be engaged. In the example of FIG. 10, the stabilizer segments 1012 may be wrapped into one or more turns 1026 and 1028 having a pre-formed diameter. For example, the stabilizer segments 1012 may be formed into spiral shapes with one or more windings or turns 1026, 1028 that are pre-disposed or biased to radially expand to a diameter sufficient to firmly fit against the interior walls of the vein.

Optionally, a single stabilizer segment 1012 may be used. Optionally, the stabilizer segment 1012 may utilize alternative shapes for stabilization, such as an S-shape, a T-shape, a Y-shape, a U-shape and the like. Optionally, the stabilizer segment 1012 may be split into multiple (e.g., 2-4) stabilizer end-segments that project outward in different directions and contact different areas of the wall tissue. The conductor wires extend from the IIMD, within the transition segment 1014 (FIG. 2) and the extension body 1007, to the electrodes 1005. In the event that the stabilization segment 1012 extends beyond an outermost electrode 1005 or 1006, the conductors would terminate at the outermost electrode 1005, 1006 such that the stabilizer segment 1012 extending beyond the outermost electrode 1005, 1006 would be void of conductor wires.

In the example of FIG. 10 the electrodes are designated 1005, 1006 to indicate that the illustrated portion of the extension body 1007 may be an intermediate portion or the end portion. The point denoted 1030 may represent the end of the extension body 1007 and be located proximate to the LV (or proximate to the LA when no LV pacing/sensing is desired). Alternatively, the stabilizer segment 1012 near 1030 may be omitted to locate electrodes 1006 at the apex of the LV for LV pacing/sensing. Alternatively, the point denoted 1030 may represent an intermediate point along the extension body 1007 with another active segment thereafter.

The active segment(s) 1010 is biased, by the stabilizer segment(s) 1012, to extend in transverse direction 1032 away from the length (or longitudinal axis 1034) of the extension body 1007 toward the LA wall and/or LV wall. The active segment(s) 1010 has a pre-formed curved shape, such as a large C-shape, or U-shape. The active segment(s) 1010 includes one or more electrodes 1005, 1006 that are provided in a trough area 1036 of the C-shape or U-shape. The electrodes 1005, 1006 are spaced apart from one another, within the trough area 1036, by an inter electrode spacing 1038. The trough area 1036 of the active segment 1010, and thus the electrodes 1005, 1006 are biased in the direction to engage a region of wall tissue of interest. For example, the electrodes 1005, 1006 may be biased to engage distal wall tissue at a distal activation site (relative to the chamber which the IIMD 1086 is implanted) within the conduction network of the LA or LV (adjacent chamber). Optionally, tines or other active fixation members may be included around the hump or trough area 1036 of the active segment 1010 in order to improve fixation as the RAA fixation mechanism.

The extension body 1007 is comprised of a flexible material having a pre-formed, memorized, permanent implanted state that is shaped to conform to select anatomical contours in the heart and to bias the active segment 1010 and stabilization arm 1012 against the wall tissue at regions of interest. One curved shape may be used for all patients. As another example, prior to implant, the patient's heart may be analyzed to identify the size of one or more chambers of interest and to identify the size and/or shape of the LA or LV. In this example, different IC device extensions 1002 may be available with different size and/or shape active segments. The physician may select the IC device extension 1002 that represents the closest match to the size/shape of the patient's chamber in which the IC device extension 1002 is to be implanted.

FIG. 11 shows a block diagram of an IIMD 1186 that is implanted in accordance with an embodiment. The IIMD 1186 may be implemented as a full-function biventricular pacemaker, equipped with both atrial and ventricular sensing and pacing circuitry for four chamber sensing and stimulation therapy (including both pacing and shock treatment). Optionally, the IIMD 1186 may provide full-function cardiac resynchronization therapy. Alternatively, the IIMD 1186 may be implemented with a reduced set of functions and components. For instance, the IIMD 1186 may be implemented without ventricular sensing and pacing.

The IIMD 1186 has a housing 1100 to hold the electronic/computing components. The housing 600 (which is often referred to as the “can”, “case”, “encasing”, or “case electrode”) may be programmably selected to act as the return electrode for certain stimulus modes. Housing 1100 further includes a connector (not shown) with a plurality of terminals 1102, 1104, 1106, 1108, and 1110. The terminals may be connected to electrodes that are located in various locations within and about the heart. For example, the terminals may include: a terminal 1102 to be coupled to a first electrode or first set of electrodes (e.g. a tip electrode or electrodes) located in or near a first chamber; a terminal 1104 to be coupled to a second electrode or second set of electrodes located in or near a second chamber; a terminal 1106 to be coupled to a third electrode or third set of electrodes located in or near the first or second chamber; terminals 1108 and 1110 to be coupled to a fourth electrode or fourth set of electrodes located in or near the a third chamber. The type and location of each electrode may vary. For example, the electrodes may include various combinations of ring, tip, coil and shocking electrodes and the like.

The IIMD 1186 includes a programmable microcontroller 1120 that controls various operations of the IIMD 1186, including cardiac monitoring and stimulation therapy. Microcontroller 1120 includes a microprocessor (or equivalent control circuitry), RAM and/or ROM memory, logic and timing circuitry, state machine circuitry, and I/O circuitry.

IMD 1186 further includes a first chamber pulse generator 1122 that generates stimulation pulses for delivery by one or more electrodes coupled thereto. The pulse generator 1122 is controlled by the microcontroller 1120 via control signal 1124. The pulse generator 1122 is coupled to the select electrode(s) via an electrode configuration switch 1126, which includes multiple switches for connecting the desired electrodes to the appropriate I/O circuits, thereby facilitating electrode programmability. The switch 1126 is controlled by a control signal 628 from the microcontroller 1120.

In the example of FIG. 11, a single pulse generator 1122 is illustrated. Optionally, the IIMD 1186 may include multiple pulse generators, similar to pulse generator 1122, where each pulse generator is coupled to one or more electrodes and controlled by the microcontroller 1120 to deliver select stimulus pulse(s) to the corresponding one or more electrodes.

Microcontroller 1120 is illustrated as including timing control circuitry 1132 to control the timing of the stimulation pulses (e.g., pacing rate, atrio-ventricular (AV) delay, atrial interconduction (A-A) delay, or ventricular interconduction (V-V) delay, etc.). The timing control circuitry 1132 may also be used for the timing of refractory periods, blanking intervals, noise detection windows, evoked response windows, alert intervals, marker channel timing, and so on. Microcontroller 1120 also has an arrhythmia detector 1134 for detecting arrhythmia conditions and a morphology detector 1136. Although not shown, the microcontroller 1120 may further include other dedicated circuitry and/or firmware/software components that assist in monitoring various conditions of the patient's heart and managing pacing therapies.

The IIMD 1186 is further equipped with a communication modem (modulator/demodulator) 1140 to enable wireless communication with the remote slave pacing unit 1106. In one implementation, the communication modem 1140 uses high frequency modulation. As one example, the modem 1140 transmits signals between a pair of electrodes of the lead assembly 1104, such as between the can 1100 and the right ventricular tip electrode 1122. The signals are transmitted in a high frequency range of approximately 20-80 kHz, as such signals travel through the body tissue in fluids without stimulating the heart or being felt by the patient.

The communication modem 1140 may be implemented in hardware as part of the microcontroller 1120, or as software/firmware instructions programmed into and executed by the microcontroller 1120. Alternatively, the modem 1140 may reside separately from the microcontroller as a standalone component.

The IIMD 1186 includes sensing circuitry 1144 selectively coupled to one or more electrodes that perform sensing operations, through the switch 1126 to detect the presence of cardiac activity in the corresponding chambers of the heart. The sensing circuit 1144 is configured to perform bipolar sensing between one pair of electrodes and/or between multiple pairs of electrodes. The sensing circuit 1144 detects NF electrical activity and rejects FF electrical activity. The sensing circuitry 1144 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the cardiac signal of interest. The automatic gain control enables the unit to sense low amplitude signal characteristics of atrial fibrillation. Switch 1126 determines the sensing polarity of the cardiac signal by selectively closing the appropriate switches. In this way, the clinician may program the sensing polarity independent of the stimulation polarity.

The output of the sensing circuitry 1144 is connected to the microcontroller 1120 which, in turn, triggers or inhibits the pulse generator 1122 in response to the absence or presence of cardiac activity. The sensing circuitry 1144 receives a control signal 1146 from the microcontroller 1120 for purposes of controlling the gain, threshold, polarization charge removal circuitry (not shown), and the timing of any blocking circuitry (not shown) coupled to the inputs of the sensing circuitry.

In the example of FIG. 11, a single sensing circuit 1144 is illustrated. Optionally, the IIMD 1186 may include multiple sensing circuit, similar to sensing circuit 1144, where each sensing circuit is coupled to one or more electrodes and controlled by the microcontroller 1120 to sense electrical activity detected at the corresponding one or more electrodes. The sensing circuit 1144 may operate in a unipolar sensing configuration or in a bipolar sensing configuration.

The IIMD 1186 further includes an analog-to-digital (ND) data acquisition system (DAS) 1150 coupled to one or more electrodes via the switch 1126 to sample cardiac signals across any pair of desired electrodes. The data acquisition system 1150 is configured to acquire intracardiac electrogram signals, convert the raw analog data into digital data, and store the digital data for later processing and/or telemetric transmission to an external device 1154 (e.g., a programmer, local transceiver, or a diagnostic system analyzer). The data acquisition system 1150 is controlled by a control signal 1156 from the microcontroller 1120.

The microcontroller 1120 is coupled to a memory 1160 by a suitable data/address bus 1162. The programmable operating parameters used by the microcontroller 1120 are stored in memory 1160 and used to customize the operation of the IIMD 1186 to suit the needs of a particular patient. Such operating parameters define, for example, pacing pulse amplitude, pulse duration, electrode polarity, rate, sensitivity, automatic features, arrhythmia detection criteria, and the amplitude, wave shape and vector of each shocking pulse to be delivered to the patient's heart 1108 within each respective tier of therapy.

The operating parameters of the IIMD 1186 may be non-invasively programmed into the memory 1160 through a telemetry circuit 1164 in telemetric communication via communication link 1166 with the external device 1154. The telemetry circuit 1164 allows intra-cardiac electrograms and status information relating to the operation of the IIMD 1186 (as contained in the microcontroller 1120 or memory 1160) to be sent to the external device 1154 through the established communication link 1166.

The IIMD 1186 can further include magnet detection circuitry (not shown), coupled to the microcontroller 1120, to detect when a magnet is placed over the unit. A magnet may be used by a clinician to perform various test functions of the unit 1186 and/or to signal the microcontroller 1120 that the external programmer 1154 is in place to receive or transmit data to the microcontroller 1120 through the telemetry circuits 1164.

The IIMD 1186 can further include one or more physiologic sensors 1170. Such sensors are commonly referred to as “rate-responsive” sensors because they are typically used to adjust pacing stimulation rates according to the exercise state of the patient. However, the physiological sensor 1170 may further be used to detect changes in cardiac output, changes in the physiological condition of the heart, or diurnal changes in activity (e.g., detecting sleep and wake states). Signals generated by the physiological sensors 1170 are passed to the microcontroller 1120 for analysis. The microcontroller 1120 responds by adjusting the various pacing parameters (such as rate, AV Delay, V-V Delay, etc.) at which the atrial and ventricular pacing pulses are administered. While shown as being included within the unit 1186, the physiologic sensor(s) 1170 may be external to the unit 1186, yet still be implanted within or carried by the patient. Examples of physiologic sensors might include sensors that, for example, sense respiration rate, pH of blood, ventricular gradient, activity, position/posture, minute ventilation (MV), and so forth.

A battery 1172 provides operating power to all of the components in the IIMD 1186. The battery 1172 is capable of operating at low current drains for long periods of time, and is capable of providing high-current pulses (for capacitor charging) when the patient requires a shock pulse (e.g., in excess of 2 A, at voltages above 2 V, for periods of 10 seconds or more). The battery 1172 also desirably has a predictable discharge characteristic so that elective replacement time can be detected. As one example, the unit 1186 employs lithium/silver vanadium oxide batteries.

The IIMD 1186 further includes an impedance measuring circuit 1174, which can be used for many things, including: lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves; and so forth. The impedance measuring circuit 1174 is coupled to the switch 1126 so that any desired electrode may be used. The microcontroller 1120 further controls a shocking circuit 1180 by way of a control signal 1182. The shocking circuit 1180 generates shocking pulses of low (e.g., up to 0.5 joules), moderate (e.g., 0.5-10 joules), or high energy (e.g., 10 to 40 joules), as controlled by the microcontroller 1120.

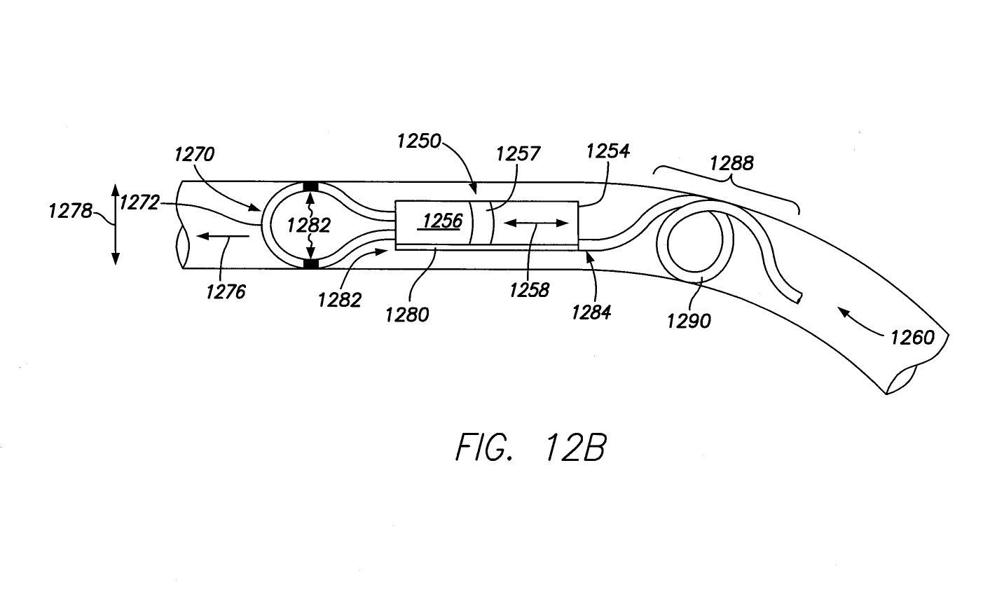

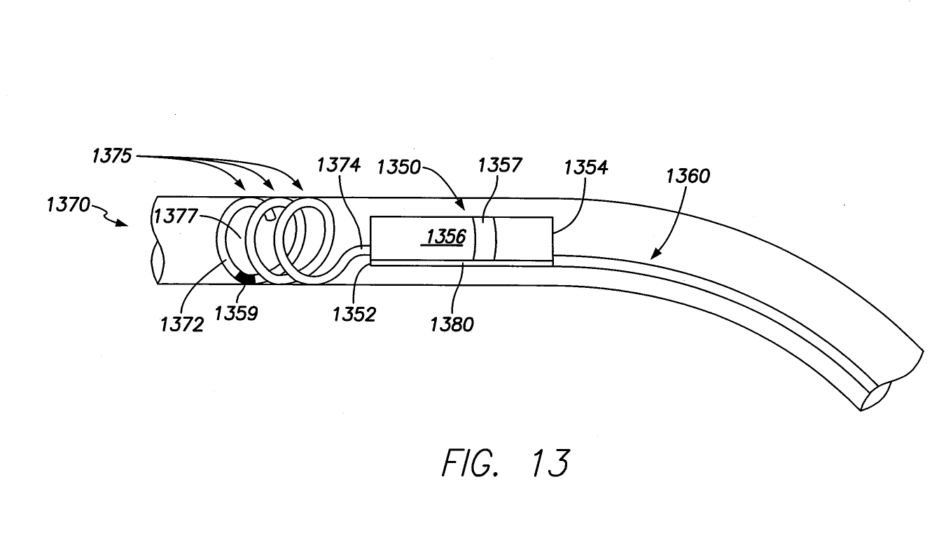

FIG. 12A illustrates an IIMD 1250 formed in accordance with an alternative embodiment. The IIMD 1250 is shown partially loaded into the sheath 1248 and partially extending from the distal end 1246 of the sheath 1248. The IIMD 1250 has a distal end 1252 and a proximal end 1254 located at opposite ends of a housing 1256. The housing may be generally cylindrically shaped extending along a longitudinal axis 1256.

An IC device extension (ICDE) 1260 is electrically and physically coupled to the proximal end 1256. The ICDE extends outward from the housing 1256 along a vessel of interest. The ICDE 1260 has a proximal end 1262 that may be permanently or removably attached to the housing 1256. The ICDE 1260 includes an extension lumen 1254 extending along a length of the ICDE 1260. The extension lumen 1264 is configured to receive a placement tool 1266 during implant and maneuvering of the ICDE 1260 to a position of interest at which sensing and stimulation may be delivered to desired chambers of the heart.

A stabilization segment 1270 is coupled to the distal end 1252 of the housing 1256. The stabilizer segment 1270 may include various forms. In the example of FIG. 12A, the stabilizer segment 1270 is formed with a looped body 1272 that has loop ends 1274 permanently or removably attached to the distal end 1252 of the IIMD 1250. The looped body 1272 is compressed within the sheath 1248 and extends in a rearward direction 1276 from the IIMD 1250. By way of example, when the IIMD 1250 is implanted in the coronary sinus, the rearward direction 1276 is directed toward the ostrium and the right atrium. The looped body 1272 is formed of the flexible materials discussed herein that are continuously biased to return to an original preformed shape.

The IIMD 1250 includes at least one device lumen 1280 formed along a periphery of the housing 1256. The device lumen 1280 defines a channel or passage and has open back and front ends 1282 and 1284 to extend entirely through the housing 1256 between the distal and proximal ends 1252 and 1254. The device lumen 1280 is configured to slidably receive the placement tool 1266 which extends entirely through the device lumen 1280 as well as through the ICDE 1260.

Optionally, one or more electrodes 1257, 1259 may be provided on at least one of the stabilization segment 1270 and the housing 1256 at a first position such that, when the IIMD 1250 is implanted in the coronary sinus, the first electrode(s) 1257, 1259 is configured to engage wall tissue at a first activation site within a conduction network of a first chamber.

FIG. 12B illustrates the IIMD 1250 once the sheath 1248 has been removed and the placement tool 1266 has been withdrawn. In FIG. 12B, the IIMD 1250 is located at its final desired implant location, such as within the coronary sinus proximate to the LA. As shown in FIG. 12B, the stabilizer segment 1270 continues to project in the rearward direction 1276, as well as flared in a transverse direction 1278 relative to the longitudinal axis 1258 of the IIMD 1250. The looped body 1272 is urged outward due to its internal shape memory until passively and securely abutting and engaging the walls of the vessel. The looped body 1272 is preformed to flair in the transverse direction 1278 by a distance near or slightly larger than the estimated diameter of the vessel of interest in order to continuously apply sufficient force against the walls of the vessel to resist longitudinal shifting along the length of the vessel of interest. The stabilizer segment 1270 prevents shifting of the IIMD 1250 along the vessel of interest in this manner.

The looped body 1272 is formed of a material that will collapse and straighten when loaded into the sheath 1248 of the introducer, but then return to its preformed shape when the sheath 1248 is removed. The stabilizer segment 1270 may be formed of various materials discussed herein, including the materials used to form the ICDE 1260, as well as flexible memory materials such as certain permanent metals, magnesium based materials, iron alloys, nitynol and the like.

As shown in FIG. 12B, the placement tool 1266 of FIG. 12A has been removed from the device lumen 1280 and from the extension lumen 1264. Once the placement tool 1266 is removed from the extension lumen 1264, the ICDE 1260 is also permitted to return to its preformed shape. In the example of FIG. 12B, the ICDE 1260 includes a stabilizer segment 1288 having one or more turns 1290 that coil and expand to securely engage the wall of the vessel of interest.

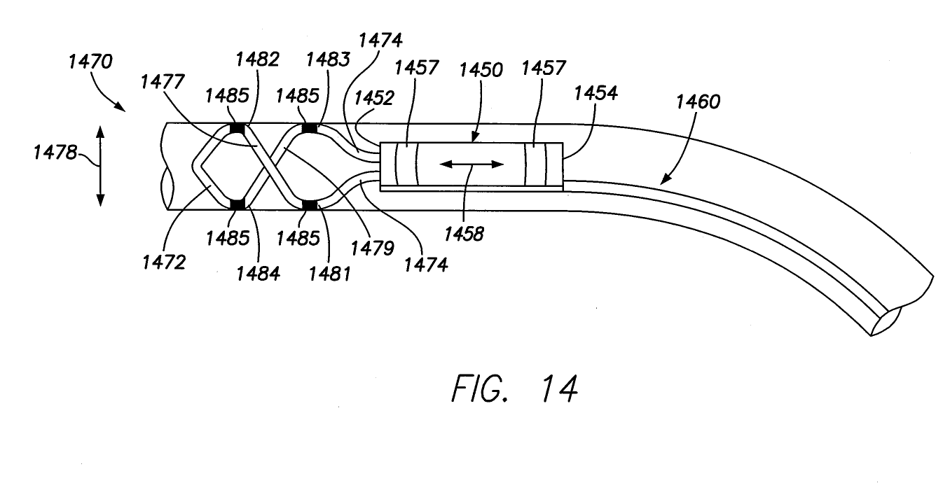

FIG. 13 illustrates an IIMD 1350 and stabilizer segment 1370 formed in accordance with an alternative embodiment. The stabilizer segment 1370 includes a body 1372 that is formed in a plurality of coils. When deployed, the coils 1375 expand outward to securely butt against the walls of the vessel of interest. The stabilizer segment 1370 includes an end 1374 that is permanently or removably secured to the distal end 1352 of the IIMD 1350. The stabilizer segment 1370 maintains the housing 1356 of the IIMD 1350 predetermined implant location. In FIG. 13, the device lumen 1380 is illustrated to be open with the placement tool removed. A portion of the ICDE 1360 is shown to extend from proximal end 1354.

As one example, the coils 1375 may be formed in a spiral manner to maintain a large open area 1377 through the coils 1375, thereby avoiding interference with the normal passage of blood through the vessel. In the example of FIG. 13, the IIMD 1350 is shown to be somewhat held within a central position within the vessel to afford a substantial amount of open area about the IIMD 1350 to avoid interference with normal blood flow. Optionally, the IIMD 1350 may be held by the stabilizing segments 1370 against a wall of the vessel of interest to afford a large passage area along a remainder of the vessel of interest.

Optionally, one or more electrodes 1357, 1359 may be provided on at least one of the stabilization segment 1370 and the housing 1356 at a first position such that, when the IIMD 1350 is implanted in the coronary sinus, the first electrode(s) 1357, 1359 is configured to engage wall tissue at a first activation site within a conduction network of a first chamber.

FIG. 14 illustrates an IIMD system 1450 formed in accordance with an alternative embodiment. The IIMD 1450 has an ICDE 1460 attached to a proximal end 1454 and a stabilizer segment 1470 attached to the distal end 1452. The system in FIG. 14 is illustrated in a deployed position with the sheath and placement tools removed. The stabilizer segment 1470 has a body 1472 that is preformed into a zigzag pattern. The body 1472 includes a plurality of legs 1477 and 1479 that are shaped to overlap in a scissor configuration with each of the legs 1477, 1479 having one or more knees or bends 1481-1484 that project outward in the transverse direction 1478 relative to a longitudinal axis 1458 of the IIMD 1450. As the knees or bends 1481-1484 press outward, the knees/bends 1481-1484 securely abut against and engage the walls of the vessel of interest.

Optionally, the stabilizer segment 1470 may include one or more active fixation elements 1485 located proximate the bends 1481-1484. As the legs 1477 and 1479 press outward, the active fixation members securely engage the wall of the vessel of interest. Optionally the active fixation members 1485 may be added to any of the stabilizing segments discussed herein, whether the stabilizing segment is a separate component extending from the IIMD or represents a segment within an IC device extension. Alternatively, the active fixation members may be entirely removed.

Optionally, one or more electrodes 1457 may be provided on at least one of the stabilization segment 1470 and the housing 1456 at a first position such that, when the IIMD 1450 is implanted in the coronary sinus, the first electrode(s) 1457, 1459 is configured to engage wall tissue at a first activation site within a conduction network of a first chamber.

In accordance with at least the embodiments of FIGS. 12-14, the introducer assembly 1270 includes a placement tool 1266 that is located within the sheath 1248 and extending through an ICDE lumen 1264 in the ICDE 1260, to at least a distal end (not shown) of the ICDE 1260. The placement tool 1266 maintaining the ICDE 1260 in an elongated collapsed state while the placement tool is within the ICDE lumen 1264. The ICDE 1260 returning to an original curved preformed shape when the placement tool 1266 is withdrawn from the ICDE lumen 1264. When the IIMD 1250 includes a device lumen 1280 through a housing 1256 of the IIMD 1250, the placement tool 1266 is advanced through the device lumen 1280 into the ICDE lumen 1266 to maintain the ICDE in the elongated collapsed state during an advancing operation. The placement tool 1266 is removed from the device lumen 1280 during the withdrawing operation.

During the method of implanting the IIMD, ICDE and stabilizer segment, the method comprises maneuvering an introducer assembly through a local chamber of a heart toward a coronary sinus, the introducer assembly including a sheath in which the IIMD, ICDE and stabilizer segment are loaded, the sheath holding at least the stabilizer segment in a compressed state; discharging the ICDE from a distal end of the sheath and maneuvering the ICDE to a first implant location such that a first electrode on the ICDE is located at a first activation site in the vessel of interest proximate to a first chamber of the heart. Next the method includes discharging the IIMD and stabilizer segment out of the sheath into the coronary sinus to a second implant location; and permitting the stabilizer segment to deploy to an original preformed shape. The stabilizer segment expands in a transverse direction relative to a longitudinal axis of the IIMD in order to securely abut against a wall of the vessel of interest in order to retain the IIMD at the second implant location. Optionally, the method may include advancing a placement tool within the sheath, through an ICDE lumen in the ICDE, to at least a distal end of the ICDE. The placement tool maintains the ICDE in an elongated collapsed state while maneuvering the ICDE to the first implant location. The method further includes withdrawing the placement tool from the ICDE lumen within the ICDE once the ICDE is at the first implant location. The ICDE returns to an original curved preformed shape when the placement tool is withdrawn. As noted above, the placement tool may be a stylet, a guide wire and the like.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

What is claimed is:

1. An assembly for introducing a device within a heart of a patient, the assembly comprising:
a sheath having at least one internal passage, wherein the sheath is configured to be maneuvered into a local chamber of the heart;
an intra-cardiac implantable medical device (IIMD) retained within the at least one internal passage, wherein the IIMD is configured to be discharged from a distal end of the sheath, the IIMD having a housing with a first active fixation member configured to anchor the IIMD at a first implant location within a local chamber of the heart, a first electrode provided on the housing at a first position such that, when the IIMD is implanted in the local chamber, the first electrode is configured to engage wall tissue at a first activation site within a conduction network of a first chamber;
an intra-cardiac (IC) device extension having a transition segment and an extension body, the transition segment electrically coupled to the IIMD housing and the extension body, the transition segment being sufficient in length to enable the extension body to be spaced apart from the housing of the IIMD and located in at least one of a coronary sinus and a tributary vein branching from the coronary sinus, the extension body being sufficient in length to extend along the at least one of the coronary sinus and tributary vein proximate to a second chamber of the heart, the extension body including an active segment configured to be positioned at a second implant location proximate to the second chamber when the extension body is located at a desired position;
a second electrode provided on the active segment of the extension body, the second electrode configured to engage wall tissue at a second activation site within the conduction network of the second chamber; and
a controller, within the housing, configured to cause stimulus pulses to be delivered through at least one of the first and second electrodes to at least one of the first and second activation sites, respectively.

2. The assembly of claim 1, wherein the sheath comprises a flexible, longitudinal, cylindrical open-ended tube defining the internal passage.

3. The assembly of claim 1, further comprising a pusher rod within the sheath, the pusher rod being removably connected to the IIMD, wherein the pusher rod is configured to push the IIMD out of the sheath and rotate the IIMD to actively attach the IIMD at the first implant location.

4. The assembly of claim 1, wherein the sheath includes first and second lumens configured to receive the IIMD and the IC device extension, respectively.

5. The assembly of claim 1, wherein the extension body of the IC device extension includes a lumen therein with an open proximal end, the assembly further comprising a placement tool at least partially received in the lumen to guide the extension body to the second implant location.

6. The assembly of claim 5, wherein the placement tool represent one of:
i) a combination of a guide wire and an ICDE pusher rod, the guide wire configured to pass through the lumen in the extension body and project beyond an open distal end of the extension body, the ICDE pusher rod having a distal end configured to abut against a proximal end of the extension body to advance the extension body to the second implant location; and
ii) a stylet the projects into the lumen in the extension body and abuts against a closed distal end of the extension body.

7. The assembly of claim 5, wherein the extension body includes a distal end having a flange thereon with a guide wire passage through the flange, the flange dimensioned to abut against and block a stylet when inserted into the lumen, the passage dimension to pass a guide wire therethrough when inserted into the lumen.

8. The assembly of claim 1, wherein the IIMD is anchored in the right atrial appendage as the first implant location and the extension body is located adjacent the left ventricle as the second implant location, the controller delivering dual chamber sensing and pacing.

9. The assembly of claim 1, wherein the IIMD is anchored in the ventricular vestibule such that the first activation site is within the conductive network of a right ventricle and the extension body is located adjacent to the left ventricle as the second implant location, the controller delivering dual chamber sensing and pacing.

10. A method of implanting an intra-cardiac implantable medical device (IIMD) having an intra-cardiac (IC) device extension, the method comprising:
maneuvering an introducer assembly into a local chamber of a heart;
pushing the IIMD out of a sheath of the introducer assembly toward a first implant location;
anchoring the IIMD at the first implant location with a first electrode located at a first activation site within a conductive network of a first chamber;
moving the sheath away from the IIMD;
maneuvering the introducer assembly into a coronary sinus toward a vessel of interest;
discharging the IC device extension out of the sheath at a second implant location such that a second electrode on the IC device extension is located at a second activation site in the vessel of interest proximate to a second chamber of the heart;
configuring a controller, within the IIMD, to cause stimulus pulses to be delivered through at least one of the first and second electrodes to at least one of the local and distal activation sites, respectively.

11. An assembly for introducing a device within a heart of a patient, the assembly comprising:
a sheath having at least one internal passage, wherein the sheath is configured to be maneuvered to a coronary sinus of the heart;
an intra-cardiac implantable medical device (IIMD) retained within the at least one internal passage, wherein the IIMD is configured to be discharged from a distal end of the sheath into the coronary sinus, the IIMD having a housing with distal and proximal ends;
a stabilizer segment joined to the proximal end of the housing, the stabilizer segment configured to retain the IIMD at a first implant location within the coronary sinus;
an intra-cardiac (IC) device extension (ICDE) having a transition segment and an extension body, the transition segment electrically coupled to the IIMD housing and the extension body, the transition segment being sufficient in length to enable the extension body to be spaced apart from the housing of the IIMD and located in at least one of the coronary sinus and a tributary vein branching from the coronary sinus, the extension body being sufficient in length to extend along the at least one of the coronary sinus and tributary vein proximate to a first chamber of the heart, the extension body including an active segment configured to be positioned at a first implant location proximate to the first chamber when the extension body is located at a desired position;
a first electrode provided on the active segment of the extension body, the first electrode configured to engage wall tissue at a first activation site within the conduction network of the first chamber; and
a controller, within the housing, configured to cause stimulus pulses to be delivered by the first electrode to the first activation site.

12. The assembly of claim 11, further comprising at least one second electrode provided on at least one of the stabilizer segment and the housing at a second position such that, when the IIMD is implanted in the coronary sinus, the second electrode is configured to engage wall tissue at a second activation site within a conduction network of a second chamber, wherein the controller is configured to cause stimulus pulses to be delivered, in a dual chamber synchronous manner, through the first and second electrodes to the first and second activation sites, respectively.

13. The assembly of claim 11, wherein the stabilizer segment is formed with a looped body that has loop ends permanently or removably attached to the distal end of the IIMD, the looped body being compressed within the sheath and extending in a rearward direction from the IIMD directed toward an ostrium and a right atrium, the looped body formed of a flexible material that is continuously biased to return to an original preformed shape.

14. The assembly of claim 11, wherein the stabilizer segment includes a body that is formed in a plurality of coils, the coils formed in a spiral manner to maintain a large open area through the coils.

15. The assembly of claim 11, wherein the stabilizer segment has a body that is preformed into a zigzag pattern, the body including a plurality of legs that are shaped to overlap in a scissor configuration with each of the legs having one or more bends that project outward in a transverse direction relative to a longitudinal axis of the IIMD, as the bends press outward, the bends securely abutting against and engaging the walls of the vessel of interest.

16. The assembly of claim 11, further comprising:
a placement tool located within the sheath and extending through an ICDE lumen in the ICDE, to at least a distal end of the ICDE, the placement tool maintaining the ICDE in an elongated collapsed state when the placement tool is inserted into the ICDE lumen, the ICDE returning to an original curved preformed shape when the placement tool is withdrawn from the ICDE.

17. The assembly of claim 11, wherein the IIMD includes a device lumen through a housing of the IIMD, the lumen extending between the proximal and distal ends, a placement tool being advanced through the device lumen into an ICDE lumen to maintain the ICDE in an elongated collapsed state during an advancing operation, the placement tool being removed from the device lumen during a withdrawing operation.

18. A method of implanting an intra-cardiac system that comprises an intra-cardiac implantable medical device (IIMD) having proximal and distal ends, an intra-cardiac device extension (ICDE) joined to the distal end, and a stabilizer segment joined to the proximal end, the method comprising:
maneuvering an introducer assembly through a local chamber of a heart toward a coronary sinus, the introducer assembly including a sheath in which the IIMD, ICDE and stabilizer segment are loaded, the sheath holding at least the stabilizer segment in a compressed state;
discharging the ICDE from a distal end of the sheath and maneuvering the ICDE to a first implant location such that a first electrode on the ICDE is located at a first activation site in the vessel of interest proximate to a first chamber of the heart;
discharging the IIMD and stabilizer segment out of the sheath into the coronary sinus to a second implant location;
permitting the stabilizer segment to deploy to an original preformed shape, the stabilizer segment expands in a transverse direction relative to a longitudinal axis of the IIMD in order to securely abut against a wall of the vessel of interest in order to retain the IIMD at the second implant location.

19. The method of claim 18, further comprising:
advancing a placement tool within the sheath, through an ICDE lumen in the ICDE, to at least a distal end of the ICDE, the placement tool maintaining the ICDE in an elongated collapsed state while maneuvering the ICDE to the first implant location; and
withdrawing the placement tool from the ICDE lumen within the ICDE once the ICDE is at the first implant location, the ICDE returning to an original curved preformed shape when the placement tool is withdrawn.

20. The method of claim 18, further comprising configuring a controller, within the IIMD, to cause stimulus pulses to be delivered through the first electrode to the first activation site.
